(19) United States
(12) Patent Application Publication
(10) Pub. No.: US 2004/0101566 A1
(43) Pub. Date: May 27, 2004

(54) Novel benzoyl peroxide compositions

(75) Inventors: Eugene R. Cooper, Berwyn, PA (US); Christian F. Wertz, Brookhaven, PA (US)

Correspondence Address: ELAN DRUG DELIVERY, INC., C/O FOLEY & LARDNER, 3000 K STREET, N.W., SUITE 500, WASHINGTON, DC, 20007-5109, US

(73) Assignee: Elan Pharma International Limited

(21) Appl. No.: 10/677,857

(22) Filed: Oct. 3, 2003

Related U.S. Application Data
(63) Continuation-in-part of application No. 10/357,514, filed on Feb. 4, 2003.
(60) Provisional application No. 60/353,230, filed on Feb. 4, 2002.

(51) Int. Cl. A61K 9/14; A61K 31/192

(52) U.S. Cl. 424/489; 514/568

(57) ABSTRACT

The present invention is directed to nanoparticulate compositions comprising benzoyl peroxide. The benzoyl peroxide particles of the composition have an effective average particle size of less than about 2 microns.

### RELATED APPLICATIONS

This application is a continuation-in-part of U.S. application Ser. No. 10/357,514, filed on Feb. 4, 2003, which claims benefit of U.S. Application No. 60/353,230, filed on Feb. 4, 2002.

### FIELD OF THE INVENTION

The present invention relates to a novel compositions of benzoyl peroxide, comprising benzoyl peroxide particles having an effective average particle size of less than about 2000 nm and at least one surface stabilizer that is preferably adsorbed to or associated with the surface of the benzoyl peroxide particles.

### BACKGROUND OF THE INVENTION

A. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having associated with the surface thereof a non-crosslinked surface stabilizer. The '684 patent does not describe nanoparticulate compositions of benzoyl peroxide.

Methods of making nanoparticulate active agent compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.” These patents do not describe methods of making nanoparticulate benzoyl peroxide.

Nanoparticulate active agent compositions are also described, for example, in U.S. Pat. Nos. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Inaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate,” U.S. Pat. No. 6,428,814 for “Bioadhesive nanoparticulate compositions having cationic surface stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract,” and U.S. Pat. No. 6,592,903 for “Nanoparticulate Dispersions Comprising a Synergistic Combination of a Polymeric Surface Stabilizer and Dioctyl Sodium Sulfosuccinate,” all of which are specifically incorporated by reference. In addition, U.S. Patent Application No. 20020012675 A1, published on Jan. 31, 2002, for “Controlled Release Nanoparticulate Compositions,” and WO 02/098565 for “System and Method for Milling Materials,” describe nanoparticulate active agent compositions, and are specifically incorporated by reference. None of these references describe nanoparticulate compositions of benzoyl peroxide.

Amorphous small particle compositions are described, for example, in U.S. Pat. Nos. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” These references do not describe nanoparticulate benzoyl peroxide.

B. Background Regarding Benzoyl Peroxide

Benzoyl peroxide is a keratolytic agent with antibacterial properties primarily used medicinally to treat acne but is also used to treat other conditions, such as decubital ulcers (bed sores) and stasis ulcers.

The mechanism of action of benzoyl peroxide in humans has not been established, but its antibacterial activity against Propionibacterium acnes may be a reason for its clinical efficacy. Patients treated with topical compositions of benzoyl peroxide show a reduction in lipids and free fatty acids in addition to a mild desquamation (drying and peeling activity). Benzoyl peroxide is indicated for treatment of acne vulgaris and may also be used to treat other conditions, such as cutaneous ulcers, based on the experience of treating physicians.

Benzoyl peroxide is a white crystalline powder that melts at 104 to 106° C. and that may explode during heating or when exposed to shock or friction. The compound is also referred to as dibenzoyl peroxide, dibenzoyl superoxide, and a variety of trade names. Benzoyl peroxide is sparingly soluble in water and alcohol and soluble in benzene, chloroform, and ether. See The Merck Index, 11^{th} Edition, p. 1124 (Merck & Co., Inc., Rahway N.J., 1989). Benzoyl peroxide has the chemical formula C₁₄H₁₀O₄ and the following chemical structure:

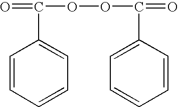

Benzoyl peroxide has been shown to be absorbed by the skin where it is converted to benzoic acid. Less than 2% of the dose enters systemic circulation as benzoic acid, which is excreted in the urine as benzoate. It is suggested that the lipophilic nature of benzoyl peroxide acts to concentrate the compound into the lipid-rich sebaceous folicle. There is no evidence of systemic toxicity caused by benzoyl peroxide in humans. Physicians'Desk Reference, 57^{th} Edition, pp. 1215 and 1927.

Benzoyl peroxide is contraindicated in individuals who have shown hypersensitivity to the compound. In addition, patients should not apply benzoyl peroxide or compositions including benzoyl peroxide to sunburned skin, to open wounds, or in combination with other harsh skin products unless directed to do so be a physician. The teratogenic effects of benzoyl peroxide are unknown, but some animal studies have suggested that benzoyl peroxide may be a tumor promoter. It is also unknown whether benzoyl peroxide is excreted in human milk.

The most frequent reactions to benzoyl peroxide applied topically are redness, drying or peeling of skin, sunburn, erythema, pruritis, dermatitis, mild stinging, or a feeling of warmth. In rare cases or in the event of an overdose, adverse reactions may include burning, itching, scaling, blistering, crusting, or swelling.

A number of drugs for topical delivery containing benzoyl peroxide as an active ingredient are currently approved for use by the U.S. Food and Drug Administration. Examples of such drugs include BenzaClin® (benzoyl peroxide and clindamycin phosphate; Dermik Laboratories, Berwyn, Pa.), BenzaGel® (benzoyl peroxide; Dermik Laboratories); Benzamycin® Pak (erythromycin and benzoyl peroxide; Dermik Laboratories); Brevoxyl®-4 Gel, Cleansing Lotion, and Creamy Wash, and Brevoxyl®-8 Gel, Cleansing Lotion, and Creamy Wash (benzoyl peroxide; Stiefel Laboratories, Inc.; Coral Gables, Fla.); Clinac™ BPO (benzoyl peroxide; Ferndale, Laboratories, Inc., Ferndale, Mich.); Duac® (benzoyl peroxide and clindamycin phosphate); and Triaz (benzoyl peroxide; Medicis, The Dermatology Co., Scotsdale, Ariz.). A large number of over the counter acne treatments and skin products contain benzoyl peroxide as an active ingredient, including products such as PanOxyl® and BenzaGel®.

U.S. Pat. No. 6,419,913, for “Topical delivery systems for active agents” describes compositions for topical administration comprising an active agent and a non-ionic lipid/surfactant-containing formulation as an enhancing agent. The active agent can be benzoyl peroxide. The non-ionic lipid, the vehicle solution, and the active agent are combined to produce solubilized drug particle sizes in the range of about 1 nm to about 500 nm. In one preferred embodiment wherein the composition contains both a polyol and an alcohol at a cumulative concentration that is less than or about equal to that of the hydrophilic component, the resulting composition is micellar in nature. One disadvantage of this type of formulation is that it requires solubilization of the benzoyl peroxide. The solubilizing agent can be an irritant, and may contribute to adverse reactions to the dosage form.

U.S. Pat. No. 6,113,921 for “Topical and transdermal delivery system utilizing submicron oil spheres” is directed to a delivery system comprising droplets in the sub-micron size range of a water-insoluble drug, such as benzoyl peroxide, in an aqueous dispersion system, wherein the droplets consist essentially of an oily liquid comprising the drug, an emulsifier and a non-ionic surfactant. Again, a disadvantage of this type of formulation is that it requires solubilization of the benzoyl peroxide. The solubilizing agent can be an irritant, and may contribute to adverse reactions to the dosage form.

U.S. Pat. No. 5,894,019 for “Topically applied pharmaceutical composition, method of preparing it and its use” is directed to a a topically applicable pharmaceutical composition comprising (a) at least one liquid lipid, (b) at least one pharmaceutically active ingredient, such as benzoyl peroxide, which is soluble in at least one of the liquid lipids and is resorbed by the skin, and (c) a hydrous gel, wherein all the active ingredients are present in a dissolved form. Again, a disadvantage of this type of formulation is that it requires solubilization of the benzoyl peroxide. The solubilizing agent can be an irritant, and may contribute to adverse reactions to the dosage form.

There is a need in the art for benzoyl peroxide compositions which can decrease frequency of dosing, improve clinical efficacy, and potentially reduce side effects. The present invention satisfies these needs.

### SUMMARY OF THE INVENTION

The present invention relates to nanoparticulate compositions comprising benzoyl peroxide. The compositions comprise benzoyl peroxide and at least one surface stabilizer preferably adsorbed on or associated with the surface of the benzoyl peroxide particles. The nanoparticulate benzoyl peroxide particles have an effective average particle size of less than about 2 microns.

Another aspect of the invention is directed to pharmaceutical compositions comprising a nanoparticulate benzoyl peroxide composition of the invention. The pharmaceutical compositions preferably comprise benzoyl peroxide, at least one surface stabilizer, and at least one pharmaceutically acceptable carrier, as well as any desired excipients. Advantages and properties of the compositions of the invention are described herein.

The invention further discloses a method of making a nanoparticulate benzoyl peroxide composition. Such a method comprises contacting benzoyl peroxide and at least one surface stabilizer for a time and under conditions sufficient to provide a nanoparticulate benzoyl peroxide composition. The one or more surface stabilizers can be contacted with benzoyl peroxide either before, preferably during, or after size reduction of the benzoyl peroxide.

The present invention is also directed to methods of treatment of cutaneous disorders using the nanoparticulate benzoyl peroxide compositions of the invention.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to nanoparticulate compositions comprising benzoyl peroxide. The compositions comprise benzoyl peroxide and at least one surface stabilizer that is preferably adsorbed on or associated with the surface of the benzoyl peroxide particles. The nanoparticulate benzoyl peroxide particles have an effective average particle size of less than about 2 microns.

As taught in the '684 patent, not every combination of surface stabilizer and active agent will result in a stable nanoparticulate composition. It was surprisingly discovered that stable nanoparticulate benzoyl peroxide formulations can be made.

The current formulations of benzoyl peroxide suffer from the following problems: (1) dosing must often be repeated several times each day; and (2) a wide variety of side effects are associated with the current dosage forms of the drug.

The present invention overcomes problems encountered with the prior art benzoyl peroxide formulations. Specifically, the nanoparticulate benzoyl peroxide formulations of the invention may offer the following advantages: (1) faster onset of action; (2) a potential decrease in the frequency of dosing; (3) smaller doses of benzoyl peroxide required to obtain the same pharmacological effect; (4) improved performance characteristics, such as higher dose loading; (5) bioadhesive benzoyl peroxide formulations, which can coat the desired site of application and be retained for a period of time, thereby increasing the efficacy of the drug as well as eliminating or decreasing the frequency of dosing; (6) low viscosity liquid nanoparticulate benzoyl peroxide compositions, useful for topical application of liquid washes; (7) the nanoparticulate benzoyl peroxide compositions can be formulated in a dried form which readily redisperses, such as for reconstitution in a liquid to be used in a wash; (8) the nanoparticulate benzoyl peroxide compositions can be used in conjunction with other active agents; and (9) the nanoparticulate benzoyl peroxide compositions do not require organic solvents or pH extremes.

Any pharmaceutically acceptable dosage form for topical administration can be utilized. Exemplary dosage forms include, but are not limited to, liquid dispersions, powders, sprays, ointments, creams, gels, lotions, liquid washes, etc. The dosage form can be, for example, a controlled release dosage form, delayed release dosage form, extended release dosage form, pulsatile release dosage form, mixed immediate release and controlled release dosage form, or a combination thereof.

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

“Conventional” or “non-nanoparticulate active agent” shall mean an active agent which is solubilized or which has an effective average particle size of greater than about 2 microns. Nanoparticulate active agents as defined herein have an effective average particle size of less than about 2 microns.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” as used herein refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Poorly water soluble drugs” as used herein means those having a solubility of less than about 30 mg/ml, preferably less than about 20 mg/ml, preferably less than about 10 mg/ml, or preferably less than about 1 mg/ml. Such drugs tend to be eliminated from the gastrointestinal tract before being absorbed into the circulation.

As used herein with reference to stable drug particles, ‘stable’ includes, but is not limited to, one or more of the following parameters: (1) that the benzoyl peroxide particles do not appreciably flocculate or agglomerate due to interparticle attractive forces, or otherwise significantly increase in particle size over time; (2) that the physical structure of the benzoyl peroxide particles is not altered over time, such as by conversion from an amorphous phase to crystalline phase; (3) that the benzoyl peroxide particles are chemically stable; and/or (4) where the benzoyl peroxide has not been subject to a heating step at or above the melting point of the benzoyl peroxide in the preparation of the nanoparticles of the invention.

‘Therapeutically effective amount’ as used herein with respect to a drug dosage, shall mean that dosage that provides the specific pharmacological response for which the drug is administered in a significant number of subjects in need of such treatment. It is emphasized that ‘therapeutically effective amount,’ administered to a particular subject in a particular instance will not always be effective in treating the diseases described herein, even though such dosage is deemed a ‘therapeutically effective amount’ by those skilled in the art. It is to be further understood that drug dosages are, in particular instances, measured as oral dosages, or with reference to drug levels as measured in blood.

A. Preferred Characteristics of the Nanoparticulate Benzoyl Peroxide Compositions of the Invention

1. Frequency of Dosing and Dosage Quantity

The benzoyl peroxide compositions of the invention may be administered less frequently and at lower doses in dosage forms such as liquid dispersions, powders, sprays, ointments, creams, gels, lotions, etc. Exemplary types of formulations useful in the present invention include, but are not limited to, liquid dispersions, powders, sprays, ointments, creams, gels, lotions, liquid washes, etc., of nanoparticulate benzoyl peroxide. Lower dosages can be used because the small particle size of the benzoyl peroxide particles ensure greater absorption, and in the case of bioadhesive nanoparticulate benzoyl peroxide compositions, the benzoyl peroxide is retained at the desired site of application for a longer period of time as compared to conventional benzoyl peroxide dosage forms.

A descreased dose of nanoparticulate benzoyl peroxide, which exhibits the same effectiveness of larger doses of conventional benzoyl peroxide, is particularly beneficial because benzoyl peroxide can be irritating and drying to the skin. Lower doses of the drug would result in decreased irritation.

In one embodiment of the invention, the therapeutically effective amount of the nanoparticulate benzoyl peroxide compositions is ⅙, ⅕, ¼, ⅓^{rd}, or ½ of the therapeutically effective amount of a non-nanoparticulate benzoyl peroxide composition.

Such lower doses are preferred as they may decrease or eliminate adverse effects of the drug. In addition, such lower doses decrease the cost of the dosage form and may increase patient compliance due to the reduction or decrease in severity of adverse reactions.

2. Redispersibility Profiles of the Nanoparticulate Benzoyl Peroxide Compositions of the Invention

An additional feature of the nanoparticulate benzoyl peroxide compositions of the invention is that the compositions redisperse such that the effective average particle size of the redispersed benzoyl peroxide particles is less than about 2 microns. This is significant, as if upon redispersion the nanoparticulate benzoyl peroxide particles present in the compositions of the invention did not redisperse to a substantially nanoparticulate particle size, then the dosage form may lose the benefits afforded by formulating benzoyl peroxide into a nanoparticulate particle size.

This is because nanoparticulate benzoyl peroxide compositions benefit from the small particle size of benzoyl peroxide; if the nanoparticulate benzoyl peroxide particles do not redisperse into the small particle sizes upon administration, then “clumps” or agglomerated benzoyl peroxide particles are formed. With the formation of such agglomerated particles, the bioavailability of the dosage form may fall.

Thus, the nanoparticulate benzoyl peroxide compositions of the invention can be formulated into a dry powder for reconstitution in a liquid media for use as a topical liquid wash.

In other embodiments of the invention, the redispersed benzoyl peroxide particles of the invention have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

3. Bioadhesive Nanoparticulate Benzoyl Peroxide Compositions

Bioadhesive nanoparticulate benzoyl peroxide compositions of the invention comprise at least one cationic surface stabilizer, which are described in more detail below. Bioadhesive formulations of benzoyl peroxide exhibit exceptional bioadhesion to biological surfaces, such as skin. This is particularly desirable for a drug to be applied topically, as the bioadhesive feature will result in fewer applications of the drug.

In the case of bioadhesive nanoparticulate benzoyl peroxide compositions, the term “bioadhesion” is used to describe the adhesion between the nanoparticulate benzoyl peroxide compositions and a biological substrate (i.e., skin). See e.g., U.S. Pat. No. 6,428,814 for “Bioadhesive Nanoparticulate Compositions Having Cationic Surface Stabilizers,” which is specifically incorporated by reference.

The bioadhesive benzoyl peroxide compositions of the invention are useful in any situation in which it is desirable to apply the compositions to a biological surface. The bioadhesive benzoyl peroxide compositions preferably coat the targeted surface in a continuous and uniform film which is invisible to the naked human eye.

4. Low Viscosity Topical Liquid Nanoparticulate Benzoyl Peroxide Compositions

A liquid dosage form of a conventional microcrystalline or no-nanoparticulate benzoyl peroxide composition would be expected to be a relatively large volume, highly viscous substance which would not be well accepted by patient populations for topical application.

Liquid dosage forms of the nanoparticulate benzoyl peroxide compositions of the invention provide significant advantages over a liquid dosage form of a conventional benzoyl peroxide microcrystalline compound. The low viscosity and silky texture of liquid dosage forms of the nanoparticulate benzoyl peroxide compositions of the invention result in advantages in both preparation and use. These advantages include, for example: (1) better subject compliance due to the perception of a lighter formulation which is easier to topically apply; (2) ease of dispensing as compared to a highly viscous formulation; (3) potential for formulating a higher concentration of benzoyl peroxide resulting in a smaller dosage volume and thus less volume for the subject to apply; and (4) easier overall formulation concerns.

The viscosities of liquid dosage forms of nanoparticulate benzoyl peroxide according to the invention are preferably less than about 1/200, less than about 1/175, less than about 1/150, less than about 1/125, less than about 1/100, less than about 1/75, less than about 1/50, or less than about 1/25 of a topical liquid dosage form of a non-nanoparticulate benzoyl peroxide composition, at about the same concentration per ml of benzoyl peroxide.

Typically the viscosity of liquid nanoparticulate benzoyl peroxide dosage forms of the invention, at a shear rate of 0.1 (1/s), is from about 2000 mPa·s to about 1 mPa s, from about 1900 mPa·s to about 1 mPa·s, from about 1800 mPa·s to about 1 mPa·s, from about 1700 mPa·s to about 1 mPa·s, from about 1600 mPa·s to about 1 mPa·s, from about 1500 mPa·s to about 1 mPa·s, from about 1400 mPa·s to about 1 mPa·s, from about 1300 mPa·s to about 1 mPa·s, from about 1200 mPa·s to about 1 mPa·s, from about 1100 mPa·s to about 1 mPa·s, from about 1000 mPa·s to about 1 mPa·s, from about 900 mPa·s to about 1 mPa·s, from about 800 mPa·s to about 1 mPa·s, from about 700 mPa·s to about 1 mPa·s, from about 600 mPa·s to about 1 mPa·s, from about 500 mPa·s to about 1 mPa·s, from about 400 mPa·s to about 1 mPa·s, from about 300 mPa·s to about 1 mPa·s, from about 200 mPa·s to about 1 mPa·s, from about 175 mPa·s to about 1 mPa·s, from about 150 mPa·s to about 1 mPa·s, from about 125 mPa·s to about 1 mPa·s, from about 100 mPa·s to about 1 mPa·s, from about 75 mPa·s to about 1 mPa·s, from about 50 mPa·s to about 1 mPa·s, from about 25 mPa·s to about 1 mPa·s, from about 15 mPa·s to about 1 mPa·s, from about 10 mPa·s to about 1 mPa·s, or from about 5 mPa·s to about 1 mPa·s.

Viscosity is concentration and temperature dependent. Typically, a higher concentration results in a higher viscosity, while a higher temperature results in a lower viscosity. Viscosity as defined above refers to measurements taken at about 20° C. (The viscosity of water at 20° C. is 1 mPa s.) The invention encompasses equivalent viscosities measured at different temperatures.

The liquid formulations of this invention can be formulated for dosages in any volume but preferably equivalent or smaller volumes than a liquid dosage form of a non-nanoparticulate benzoyl peroxide composition.

5. Combination Active Agent Compositions

The invention encompasses the nanoparticulate benzoyl peroxide compositions of the invention formulated or co-administered with one or more non-benzoyl peroxide active agents. Methods of using such combination compositions are also encompassed by the invention. The non-benzoyl peroxide active agents can be present in a crystalline phase, an amorphous phase, a semi-crystalline phase, a semi-amorphous phase, or a mixture thereof.

The compound to be administered in combination with a nanoparticulate benzoyl peroxide composition of the invention can be formulated separately from the nanoparticulate benzoyl peroxide composition or co-formulated with the nanoparticulate benzoyl peroxide composition. Where a nanoparticulate benzoyl peroxide composition is co-formulated with a second active agent, the second active agent can be formulated in any suitable manner, such as immediate-release, rapid-onset, sustained-release, or dual-release form.

Such non-benzoyl peroxide active agents can be, for example, a therapeutic agent. A therapeutic agent can be a pharmaceutical agent, including a biologic. The active agent can be selected from a variety of known classes of drugs, including, for example, nutraceuticals, retinoic acid products, antibiotics, and sulfur/salicylic acid containing preparations. Exemplary antibiotics include, for example, clindamycin and erythromycin.

Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., arginine, isoleucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.”

Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. Dietary supplements and nutraceuticals are also disclosed in Physicians'Desk Reference for Nutritional Supplements, 1st Ed. (2001) and The Physicians' Desk Reference for Herbal Medicines, 1st Ed. (2001), both of which are also incorporated by reference. A nutraceutical or dietary supplement, also known as a phytochemical or functional food, is generally any one of a class of dietary supplements, vitamins, minerals, herbs, or healing foods that have medical or pharmaceutical effects on the body.

B. Benzoyl Peroxide Compositions

The invention provides compositions comprising nanoparticulate benzoyl peroxide particles and at least one surface stabilizer. The surface stabilizers are preferably associated with the surface of the benzoyl peroxide particles. Surface stabilizers useful herein do not chemically react with the benzoyl peroxide particles or itself. Preferably, individual molecules of the surface stabilizer are essentially free of intermolecular cross-linkages. The compositions can comprise two or more surface stabilizers.

The present invention also includes nanoparticulate benzoyl peroxide compositions together with one or more non-toxic physiologically acceptable carriers, adjuvants, or vehicles, collectively referred to as carriers.

1. Benzoyl Peroxide Particles

Benzoyl peroxide is a white crystalline powder that melts at 104 to 106° C. The compound is also referred to as dibenzoyl peroxide, dibenzoyl superoxide, and a variety of trade names. Benzoyl peroxide is sparingly soluble in water and alcohol and soluble in benzene, chloroform, and ether. See The Merck Index, 11^{th} Edition, p. 1124 (Merck & Co., Inc., Rahway N.J., 1989). Benzoyl peroxide has the chemical formula C₁₄H₁₀O₄ and the following chemical structure:

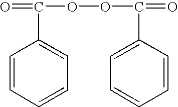

Benzoyl peroxide can be in a crystalline phase, an amorphous phase, a semi-crystalline phase, a semi-amorphous phase, or a mixtures thereof.

The mechanism of action of benzoyl peroxide in humans has not been established, but its antibacterial activity against Propionibacterium acnes may be a reason for its clinical efficacy. Patients treated with topical compositions of benzoyl peroxide show a reduction in lipids and free fatty acids in addition to a mild desquamation (drying and peeling activity). Benzoyl peroxide is indicated for treatment of acne vulgaris and may also be used to treat other conditions, such as cutaneous ulcers, based on the experience of treating physicians.

The most frequent reactions to benzoyl peroxide applied topically are redness, drying or peeling of skin, sunburn, erythema, pruritis, dermatitis, mild stinging, or a feeling of warmth. In rare cases or in the event of an overdose, adverse reactions may include burning, itching, scaling, blistering, crusting, or swelling.

2. Surface Stabilizers

The choice of a surface stabilizer for benzoyl peroxide is non-trivial and required extensive experimentation to realize a desirable formulation. Accordingly, the present invention is directed to the surprising discovery that benzoyl peroxide nanoparticulate compositions can be made.

Combinations of more than one surface stabilizer can be used in the invention. Useful surface stabilizers which can be employed in the invention include, but are not limited to, known organic and inorganic pharmaceutical excipients. Such excipients include various polymers, low molecular weight oligomers, natural products, and surfactants. Useful surface stabilizers include nonionic, anionic, cationic, zwitterionic, and ionic surfactants.

Representative examples of other useful surface stabilizers include hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-10G® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

Depending upon the desired method of administration, bioadhesive formulations of nanoparticulate benzoyl peroxide can be prepared by selecting one or more cationic surface stabilizers that impart bioadhesive properties to the resultant composition. Useful cationic surface stabilizers are described below.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt) (also known as DPPE-PEG(2000)-Amine Na) (Avanti Polar Lipids, Alabaster, Ala.), Poly(2-methacryloxyethyl trimethylammonium bromide) (Polysciences, Inc., Warrington, Pa.) (also known as S1001), poloxamines such as Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.), lysozyme, long-chain polymers such as alginic acid, carrageenan (FMC Corp.), and POLYOX (Dow, Midland, Mich.).

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric cationic surface stabilizers are any nonpolymeric compound, such as benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Preferred surface stabilizers in the nanoparticulate benzoyl peroxide compositions of the invention include, but are not limited to, lysozyme, polyvinylpyrrolidone (PVP), benzalkonium chloride (BKC), and mixtures thereof.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

The surface stabilizers are commercially available and/or can be prepared by techniques known in the art.

3. Pharmaceutical Excipients

Pharmaceutical compositions according to the invention may also comprise one or more binding agents, filling agents, lubricating agents, suspending agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Nanoparticulate Benzoyl Peroxide Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The compositions of the invention comprise benzoyl peroxide particles which have an effective average particle size of less than about 2000 nm (i.e., 2 microns), less than about 1900 nm, less than less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 140 nm, less than about 130 nm, less than about 120 nm, less than about 110 nm, less than about 100 nm, less than about 90 nm, less than about 80 nm, less than about 70 run, less than about 60 run, or less than about 50 nm, when measured by the above-noted techniques.

By “an effective average particle size of less than about 2000 nm” it is meant that at least 50% of the nanoparticulate benzoyl peroxide particles have a weight average particle size of less than about 2000 nm, when measured by the above-noted techniques. In other embodiments of the invention, at least about 70%, at least about 90%, at least about 95%, or at least about 99% of the nanoparticulate benzoyl peroxide particles have a particle size less than the effective average, by weight, i.e., less than about 2000 nm, less than about 1900 nm, less than less than about 1800 nm, less than about 1700 mm, etc.

If the nanoparticulate benzoyl peroxide composition is combined with a microparticulate benzoyl peroxide and/or non-benzoyl peroxide active agent composition, then such a composition is either solubilized or has an effective average particle size of greater than about 2 microns. By “an effective average particle size of greater than about 2 microns” it is meant that at least 50% of the microparticulate benzoyl peroxide or non-benzoyl peroxide active agent particles have a particle size greater than about 2 microns, by weight, when measured by the above-noted techniques. In other embodiments of the invention, at least about 70%, at least about 90%, at least about 95%, or at least about 99%, by weight, of the microparticulate benzoyl peroxide or non-benzoyl peroxide active agent particles have a particle size greater than about 2 microns.

In the present invention, the value for D50 of a nanoparticulate benzoyl peroxide composition is the particle size below which 50% of the benzoyl peroxide particles fall, by weight. Similarly, D90 and D99 are the particle sizes below which 90% and 99%, respectively, of the benzoyl peroxide particles fall, by weight.

5. Concentration of Nanoparticulate Benzoyl Peroxide and Surface Stabilizers

The relative amounts of benzoyl peroxide and one or more surface stabilizers can vary widely. The optimal amount of the individual components can depend, for example, upon the hydrophilic lipophilic balance (HLB), melting point, and the surface tension of water solutions of the stabilizer, etc.

The concentration of benzoyl peroxide can vary from about 99.5% to about 0.001%, from about 95% to about 0.1%, or from about 90% to about 0.5%, by weight, based on the total combined dry weight of the benzoyl peroxide and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the benzoyl peroxide and at least one surface stabilizer, not including other excipients.

C. Methods of Making Nanoparticulate Benzoyl Peroxide Formulations

The nanoparticulate benzoyl peroxide compositions can be made using, for example, milling, homogenization, or precipitation techniques. Exemplary methods of making nanoparticulate compositions are described in the '684 patent. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

Following milling, homogenization, precipitation, etc., the resultant nanoparticulate benzoyl peroxide composition can be utilized in a desired dosage formulations.

1. Milling to Obtain Nanoparticulate Benzoyl Peroxide Dispersions

Milling benzoyl peroxide to obtain a nanoparticulate dispersion comprises dispersing benzoyl peroxide particles in a liquid dispersion media in which benzoyl peroxide is poorly soluble, followed by applying mechanical means in the presence of grinding media to reduce the particle size of benzoyl peroxide to the desired effective average particle size. The dispersion media can be, for example, water, safflower oil, ethanol, t-butanol, glycerin, polyethylene glycol (PEG), hexane, or glycol. The dispersion media is preferably water.

The benzoyl peroxide particles can be reduced in size in the presence of at least one surface stabilizer. Alternatively, the benzoyl peroxide particles can be contacted with one or more surface stabilizers after attrition. Other compounds, such as a diluent, can be added to the benzoyl peroxide/surface stabilizer composition during the size reduction process. Dispersions can be manufactured continuously or in a batch mode.

2. Precipitation to Obtain Nanoparticulate Benzoyl Peroxide Compositions

Another method of forming the desired nanoparticulate benzoyl peroxide composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example:

(1) dissolving benzoyl peroxide in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Benzoyl Peroxide Nanoparticulate Compositions

Exemplary homogenization methods of preparing active agent nanoparticulate compositions are described in U.S. Pat. No. 5,510,118, for “Process of Preparing Therapeutic Compositions Containing Nanoparticles.”

Such a method comprises dispersing benzoyl peroxide particles in a liquid dispersion media in which benzoyl peroxide is poorly soluble, followed by subjecting the dispersion to homogenization to reduce the particle size of the benzoyl peroxide to the desired effective average particle size. The dispersion media can be, for example, water, safflower oil, ethanol, t-butanol, glycerin, polyethylene glycol (PEG), hexane, or glycol. The dispersion media is preferably water.

The benzoyl peroxide particles can be reduced in size in the presence of at least one surface stabilizer. Alternatively, the benzoyl peroxide particles can be contacted with one or more surface stabilizers either before or after attrition. Other compounds, such as a diluent, can be added to the benzoyl peroxide/surface stabilizer composition either before, during, or after the size reduction process. Dispersions can be manufactured continuously or in a batch mode.

D. Methods of Using Nanoparticulate Benzoyl Peroxide Formulations

The invention encompasses methods of administering to a subject in need an effective amount of a composition comprising nanoparticulate benzoyl peroxide. The benzoyl peroxide compositions of the present invention can be administered to a subject via any conventional means including, but not limited to, locally (e.g., aerosol, powders, ointments, gels, lotions, creams, or drops). As used herein, the term “subject” is used to mean an animal, preferably a mammal, including a human or non-human. The terms patient and subject may be used interchangeably.

The compositions are preferably used for treatment of dermatological and cutaneous disorders, such as treatment of acne, acne vulgaris, reduction of excessive facial oil, decubital ulcers (bed sores) and stasis ulcers.

Compositions suitable for topical delivery may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate benzoyl peroxide compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Besides such inert diluents, the composition can also include adjuvants, such as wetting agents, emulsifying and suspending agents, sweetening, flavoring, and perfuming agents.

One of ordinary skill will appreciate that effective amounts of benzoyl peroxide can be determined empirically and can be employed in pure form or, where such forms exist, in pharmaceutically acceptable salt, ester, or prodrug form. Actual dosage levels of benzoyl peroxide in the nanoparticulate compositions of the invention may be varied to obtain an amount of benzoyl peroxide that is effective to obtain a desired therapeutic response for a particular composition and method of administration. The selected dosage level therefore depends upon the desired therapeutic effect, the route of administration, the potency of the administered benzoyl peroxide, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following example is given to illustrate the present invention. It should be understood, however, that the invention is not to be limited to the specific conditions or details described in this example. Throughout the specification, any and all references to a publicly available document, including a U.S. patent, are specifically incorporated by reference.

The formulations in the examples that follow were also investigated using a light microscope. Here, “stable” nanoparticulate dispersions (uniform Brownian motion) were readily distinguishable from “aggregated” dispersions (relatively large, nonuniform particles without motion).

### EXAMPLE 1

The purpose of this example was to prepare nanoparticulate benzoyl peroxide formulations using lysozyme as a surface stabilizer.

Lysozyme, also known as muramidase, N-acetylmuramylhydrolase, and globulin G1, has a molecular weight of about 14,400. It is a mucolytic enzyme with antibiotic properties first discovered by A. Fleming, Proc. Roy. Soc. London, 93B:306 (1922). Although lysozyme has antibiotic properties, it is a large molecule that is not particularly useful as a drug. It can be applied topically, but cannot rid the entire body of disease because it is too large to travel between cells.

An aqueous dispersion of 1 wt. % lysozyme and 5 wt. % benzoyl peroxide was charged into a NanoMill™ (Elan Drug Delivery) equipped with a 10 cc batch chamber. The mill speed was maintained at 5500 rpm, the temperature was maintained at 5° C., and the mixture was milled for 1 hour. Following milling, the mean particle size, D50, and D90 for the benzoyl peroxide particles were measured using a Horiba LA-910 Static Light Scattering Particle Analyzer (Horiba Instruments, Irvine, Calif.). The milled benzoyl peroxide composition was also evaluated via a microscope to detect any aggregation. The results are shown below in Table 1.

TABLE 1
1% Lysozyme and 5% Benzoyl Peroxide

| | Mean | D50 | D90 | |
|---|---|---|---|---|
| | (nm) | (nm) | (nm) | Microscope |
| Benzoyl Peroxide | 122 | 110 | 196 | Stable |

The results demonstrate that stable nanoparticulate compositions of benzoyl peroxide can be made.

### EXAMPLE 2

The purpose of this example was to prepare nanoparticulate benzoyl peroxide formulations using lysozyme as a surface stabilizer.

An aqueous dispersion of 1 wt. % lysozyme and 5 wt. % benzoyl peroxide was charged into a DYNO®-Mill Type KDL media mill (Willy Bachofen AG, Basel, Switzerland) equipped with a 150 cc batch chamber. The mill speed was maintained at 4200 rpm, the temperature was maintained at 5° C., and the mixture was milled for 1 hour. Following milling, the mean particle size, D50, and D90 for the benzoyl peroxide particles were measured using a Horiba LA-910 Static Light Scattering Particle Analyzer (Horiba Instruments, Irvine, Calif.). The milled benzoyl peroxide composition was also evaluated via a microscope to detect any aggregation. The results are shown below in Table 1.

TABLE 1
1% Lysozyme and 5% Benzoyl Peroxide

| | Mean | D50 | D90 | |
|---|---|---|---|---|
| | (nm) | (nm) | (nm) | Microscope |
| Benzoyl Peroxide | 101 | 94 | 155 | Stable |

The results demonstrate that stable nanoparticulate compositions of benzoyl peroxide can be made.

### EXAMPLE 3

The purpose of this example was to prepare nanoparticulate benzoyl peroxide formulations using polyvinylpyrrolidone (PVP) (Plasdone™ K-29/32; BASF) and benzalkonium chloride (BKC) as surface stabilizers.

An aqueous dispersion of 1 wt. % Plasdone™ K-29/32, 0.01 wt. % BKC, and 5 wt. % benzoyl peroxide was charged into a DYNO®-Mill Type KDL media mill (Willy Bachofen AG, Basel, Switzerland) equipped with a 150 cc batch chamber. The mill speed was maintained at 4200 rpm, the temperature was maintained at 5° C., and the mixture was milled for 1 hour.

Following milling, the mean particle size, D50, and D90 for the benzoyl peroxide particles were measured using a Horiba LA-910 Static Light Scattering Particle Analyzer (Horiba Instruments, Irvine, Calif.). The milled benzoyl peroxide composition was also evaluated via a microscope to detect any aggregation. The results are shown below in Table 1.

TABLE 1
1% Plasdone K-29/32, 0.01% BKC, and 5% Benzoyl Peroxide

| | Mean | D50 | D90 | |
|---|---|---|---|---|
| | (nm) | (nm) | (nm) | Microscope |
| Benzoyl Peroxide | 205 | 196 | 283 | Stable |

The results demonstrate that stable nanoparticulate compositions of benzoyl peroxide can be made.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

We claim:

1. A composition comprising:
(a) particles of benzoyl peroxide or a salt thereof, wherein the benzoyl peroxide particles have an effective average particle size of less than about 2000 nm; and
(b) at least one surface stabilizer.

2. The composition of claim 1, wherein the benzoyl peroxide is selected from the group consisting of a crystalline phase, an amorphous phase, a semi-crystalline phase, a semi-amorphous phase, and mixtures thereof.

3. The composition of claim 1, wherein the effective average particle size of the benzoyl peroxide particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

4. The composition of claim 1 formulated into a dosage form selected from the group consisting of liquid dispersions, powders, lyophilized form, sprays, ointments, creams, gels, lotions, liquid washes, controlled release dosage form, delayed release dosage form, extended release dosage form, pulsatile release dosage form, mixed immediate release and controlled release dosage form, or a combination thereof.

5. The composition of claim 1, wherein the composition further comprises one or more pharmaceutically acceptable excipients, carriers, or a combination thereof.

6. The composition of claim 1, wherein the benzoyl peroxide or a salt thereof is present in an amount selected from the group consisting of from about 99.5% to about 0.001%, from about 95% to about 0.1%, and from about 90% to about 0.5%, by weight, based on the total combined weight of the benzoyl peroxide or a salt thereof and at least one surface stabilizer, not including other excipients.

7. The composition of claim 1, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the benzoyl peroxide or a salt thereof and at least one surface stabilizer, not including other excipients.

8. The composition of claim 1, wherein the surface stabilizer is selected from the group consisting of an anionic surface stabilizer, a cationic surface stabilizer, a zwitterionic surface stabilizer, and an ionic surface stabilizer.

9. The composition of claim 1, comprising at least two surface stabilizers.

10. The composition of claim 8, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, and random copolymers of vinyl acetate and vinyl pyrrolidone.

11. The composition of claim 8, wherein the at least one cationic surface stabilizer is selected from the group consisting of a polymer, a biopolymer, a polysaccharide, a cellulosic, an alginate, a nonpolymeric compound, and a phospholipid.

12. The composition of claim 8, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethyl-benzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIAAPOL™, ALKAQUAT™, alkyl pyridinium salts; amines, amine salts, amine oxides, imide azolinium salts, protonated quaternary acrylamides, methylated quaternary polymers, and cationic guar.

13. The composition of claim 1, wherein the surface stabilizer is lysozyme, polyvinylpyrrolidone (PVP), benzalkonium chloride (BKC), or a mixture thereof.

14. The composition of any of claims 8, 11, 12, or 13, wherein the composition is bioadhesive.

15. The composition of claim 1, additionally comprising one or more non-benzoyl peroxide active agents.

16. The composition of claim 15, wherein the additionally one or more non-benzoyl peroxide active agents are selected from the group consisting of nutraceuticals, retinoic acid, antibiotics, sulfur, and salicylic acid.

17. The composition of claim 16, wherein the antibiotic is clindamycin, erythromycin, or a combination thereof.

18. A method of making a benzoyl peroxide composition comprising contacting particles of benzoyl peroxide or a salt thereof with at least one surface stabilizer for a time and under conditions sufficient to provide a benzoyl peroxide composition having an effective average particle size of less than about 2000 nm.

19. The method of claim 18, wherein said contacting comprises grinding.

20. The method of claim 19, wherein said grinding comprises wet grinding.

21. The method of claim 18, wherein said contacting comprises homogenizing.

22. The method of claim 18, wherein said contacting comprises:
(a) dissolving particles of benzoyl peroxide or a salt thereof in a solvent;
(b) adding the resulting benzoyl peroxide solution to a solution comprising at least one surface stabilizer; and
(c) precipitating the solubilized benzoyl peroxide and surface stabilizer composition by the addition thereto of a non-solvent.

23. The method of claim 18, wherein the benzoyl peroxide or a salt thereof is selected from the group consisting of a crystalline phase, an amorphous phase, a semi-crystalline phase, a semi-amorphous phase, and mixtures thereof.

24. The method of claim 18, wherein the effective average particle size of the benzoyl peroxide particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

25. The method of claim 18, wherein the composition is formulated into a dosage form selected from the group consisting of liquid dispersions, powders, lyophilized form, sprays, ointments, creams, gels, lotions, liquid washes, controlled release dosage form, delayed release dosage form, extended release dosage form, pulsatile release dosage form, mixed immediate release and controlled release dosage form, or a combination thereof.

26. The method of claim 18, wherein the composition further comprises one or more pharmaceutically acceptable excipients, carriers, or a combination thereof.

27. The method of claim 18, wherein the benzoyl peroxide or a salt thereof is present in an amount selected from the group consisting of from about 99.5% to about 0.001%, from about 95% to about 0.1%, and from about 90% to about 0.5%, by weight, based on the total combined weight of the benzoyl peroxide or a salt thereof and at least one surface stabilizer, not including other excipients.

28. The method of claim 18, wherein the at least one surface stabilizer is present in an amount selected from the group consisting of from about 0.5% to about 99.999%, from about 5.0% to about 99.9%, and from about 10% to about 99.5% by weight, based on the total combined dry weight of the benzoyl peroxide or a salt thereof and at least one surface stabilizer, not including other excipients.

29. The method of claim 18, wherein the surface stabilizer is selected from the group consisting of an anionic surface stabilizer, a cationic surface stabilizer, a zwitterionic surface stabilizer, and an ionic surface stabilizer.

30. The method of claim 18, comprising at least two surface stabilizers.

31. The method of claim 29, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

32. The method of claim 29, wherein the at least one cationic surface stabilizer is selected from the group consisting of a polymer, a biopolymer, a polysaccharide, a cellulosic, an alginate, a nonpolymeric compound, and a phospholipid.

33. The method of claim 29, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

34. The method of claim 29, wherein the surface stabilizer is lysozyme, polyvinylpyrrolidone (PVP), benzalkonium chloride (BKC), or a mixture thereof.

35. The method of any of claims 29, 32, 33, or 34, wherein the composition is bioadhesive.

36. A method of treating a subject in need comprising administering to the subject an effective amount of a composition comprising:
(a) particles of a benzoyl peroxide or a salt thereof, wherein the benzoyl peroxide particles have an effective average particle size of less than about 2000 nm; and
(b) at least one surface stabilizer.

37. The method of claim 36, wherein the benzoyl peroxide or a salt thereof is selected from the group consisting of a crystalline phase, an amorphous phase, a semi-crystalline phase, a semi-amorphous phase, and mixtures thereof.

38. The method of claim 36, wherein the effective average particle size of the benzoyl peroxide particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

39. The method of claim 36, wherein the composition is formulated into a dosage form selected from the group consisting of liquid dispersions, powders, lyophilized form, sprays, ointments, creams, gels, lotions, liquid washes, controlled release dosage form, delayed release dosage form, extended release dosage form, pulsatile release dosage form, mixed immediate release and controlled release dosage form, or a combination thereof.

40. The method of claim 36, wherein the composition further comprises one or more pharmaceutically acceptable excipients, carriers, or a combination thereof.

41. The method of claim 36, wherein the benzoyl peroxide or a salt thereof is present in an amount selected from the group consisting of from about 99.5% to about 0.001%, from about 95% to about 0.1%, and from about 90% to about 0.5%, by weight, based on the total combined weight of the benzoyl peroxide or a salt thereof and at least one surface stabilizer, not including other excipients.

42. The method of claim 36, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the benzoyl peroxide or a salt thereof and at least one surface stabilizer, not including other excipients.

43. The method of claim 36, wherein the surface stabilizer is selected from the group consisting of an anionic surface stabilizer, a cationic surface stabilizer, a zwitterionic surface stabilizer, and an ionic surface stabilizer.

44. The method of claim 36, comprising utilizing at least two surface stabilizers.

45. The method of claim 43, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

46. The method of claim 43, wherein the at least one cationic surface stabilizer is selected from the group consisting of a polymer, a biopolymer, a polysaccharide, a cellulosic, an alginate, a nonpolymeric compound, and a phospholipid.

47. The method of claim 43, wherein the surface stabilizer is selected from the group consisting of benzalkonium chloride, polymethylmethacrylate trimethylammonium bromide, polyvinylpyrrolidone-2-dimethylaminoethyl methacrylate dimethyl sulfate, hexadecyltrimethyl ammonium bromide, cationic lipids, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethyl-benzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIAAPOL™, ALKAQUAT™, alkyl pyridinium salts; amines, amine salts, amine oxides, imide azolinium salts, protonated quaternary acrylamides, methylated quaternary polymers, and cationic guar.

48. The method of claim 36, wherein the surface stabilizer is lysozyme, polyvinylpyrrolidone (PVP), benzalkonium chloride (BKC), or a mixture thereof.

49. The method of any of claims 43, 46, 47, or 48, wherein the composition is bioadhesive.

50. The method of claim 36, additionally comprising administering one or more non-benzoyl peroxide active agents.

51. The method of claim 50, wherein the additional one or more non-benzoyl peroxide active agents are selected from the group consisting of nutraceuticals, retinoic acid, antibiotics, sulfur, and salicylic acid.

52. The composition of claim 16, wherein the antibiotic is clindamycin, erythromycin, or a combination thereof.

53. The method of claim 36, wherein the subject is a human.

54. The method of claim 36 used to treat dermatological or cutaneous conditions or disorders.

55. The method of claim 36 used to treat a condition selected from the group consisting of acne, acne vulgaris, reduction of excessive facial oil, decubital ulcers, and stasis ulcers.
